(19) United States
(12) Patent Application Publication
(10) Pub. No.: US 2023/0292688 A1
(43) Pub. Date: Sep. 21, 2023

(54) METHODS TO DECREASE SOYBEAN PLANT SEED WEIGHT

(71) Applicant: INARI AGRICULTURE TECHNOLOGY, INC., Cambridge, MA (US)

(72) Inventors: AMBER BASSETT, West Lafayette, IN (US); RODRIGO GERMAN SALA, Wildwood, MO (US)

(21) Appl. No.: 18/186,541

(22) Filed: Mar. 20, 2023

Related U.S. Application Data
(60) Provisional application No. 63/269,655, filed on Mar. 21, 2022.

(51) Int. Cl. A01H 5/10; A01H 6/54

(52) U.S. Cl. CPC A01H 5/10; A01H 6/542

(57) ABSTRACT

The present disclosure provides methods to obtain soybean plants that produce seed having decreased soybean seed weight.

### CROSS REFERENCE TO RELATED APPLICATIONS

This non-provisional U.S. patent application claims the benefit of U.S. provisional patent application Ser. No. 63/269,655, filed Mar. 21, 2022.

### BACKGROUND

Soybean is an important crop globally due to its ability to fix atmospheric nitrogen in the soil while serving as a major source of animal feed protein and soybean oil for food, industrial and biodiesel use. Soybean farmers work hard to help ensure shelves are stocked and families are fed around the world.

Farming to preserve soil, water and nutrient resources for future generations is a priority for U.S. farmers, breeders and policymakers. Unprecedented changes in farming technologies and methods in the 20th century provided food, feed and fuel to a rapidly growing world population. In the 21st century, necessity to improve soil nutrition, biodiversity and carbon sequestration, water use and pollution levels, nitrogen and phosphorus management, and land conservation drives further opportunity for advancement.

Soybean has limited genetic diversity since most varieties are found to be selected from the same original group of progenitors. The limitation of genetic resources is a major challenge for soybean improvement to overcome significant constraints for farming and production caused by climate change, reduced agricultural land availability and increased biotic and abiotic stresses. Improved technologies are necessary to break through the bottleneck to achieve further improvement in soybean agronomic traits and to guarantee yield increases to satisfy future demands for soybean the global market.

The present disclosure seeks to meet these needs.

### DESCRIPTION

One aspect of the disclosure is a method to obtain a soybean plant having decreased soybean seed weight by: (a) crossing a first soybean plant having a first seed weight with a second, different soybean plant having a second seed weight that is lower than the first seed weight to produce F1 hybrid soybean seed; (b) crossing a plant grown from the F1 hybrid soybean seed with itself to produce seed of a progeny plant of a subsequent generation; (c) growing a progeny plant of a subsequent generation from the seed of the progeny plant of the subsequent generation and crossing the progeny plant of the subsequent generation with itself to produce a progeny plant of a further subsequent generation; and, (d) repeating steps (b) and (c) using the progeny plant of the further subsequent generation from step (c) in place of the plant grown from the F1 soybean seed in step (b), where steps (b) and (c) are repeated with sufficient inbreeding and selection to produce an elite inbred soybean plant having decreased seed weight compared to the first seed weight. Seed weight includes, but is not limited to 100 seed weight, 1,000 seed weight, average single seed weight and seeds per pound. In aspects of the disclosure, decreased seed weight refers to a statistically significant decreased seed weight. In aspects of the disclosure, the second, different soybean plant does not comprise a naturally occurring mutation in an endogenous soybean gene which confers the decreased seed weight. In aspects of the disclosure, the second, different soybean plant does not comprise a loss-of-function mutation in the soybean JAG1 and/or JAG2 gene.

In another aspect, steps (b) and (c) are repeated with sufficient inbreeding and selection to produce an elite inbred soybean plant having a decreased seed weight compared to the second seed weight.

In another aspect, a stigma on the first soybean plant is cross-pollinated with anthers from the second, different soybean plant in step (a).

In another aspect, the seed size distribution of seed on the elite inbred soybean plant and the seed size distribution of seed on the first soybean plant are not substantially different.

In another aspect, the seed size distribution of seed on the elite inbred soybean plant and the seed size distribution of seed on both the first soybean plant and the second soybean plant are not substantially different.

In another aspect, the soybean seed weight of the elite inbred soybean plant is from 1% to 10% less than the first seed weight of the first soybean plant.

In another aspect, the soybean seed weight of the elite inbred soybean plant is from 1% to 10% less than the second seed weight of the second soybean plant.

In another aspect, the selection step (d) includes phenotypic evaluation and selection for the decreased seed weight and/or similar seed size distribution.

In another aspect, the selection step (d) includes marker assisted evaluation and selection for the decreased seed weight, such as by genotyping progeny for the presence and/or absence of a molecular marker associated with seed weight.

In another aspect, the selection step (d) includes using genetic markers to compare a genetic complement of a progeny plant with a genetic complement of the first soybean variety and/or the second soybean variety.

In another aspect, the selection in step (d) is performed in at least two progeny generations selected from the group consisting of F3 progeny, F4 progeny, F5 progeny, F6 progeny, F7 progeny and F8 progeny. In other aspects, the selection step (d) is performed in at least three, four or five progeny generations.

Another aspect is a soybean plant produced by the inventive methods having decreased seed weight compared to the seed weight of the first soybean plant or the first and the second soybean plant. In other aspects, is a soybean seed, plant part or plant cell produced by the inventive methods. In other aspects, is a soybean plant part of produced by the inventive methods, where the plant part is selected from the group consisting of a leaf, pollen, embryo, root, root tip, anther, pistil, flower, seed, pod, and stem. In aspects of the disclosure, the soybean plants, parts, seeds, and cells do not comprise a naturally occurring mutation in an endogenous soybean gene which confers the decreased seed weight. In aspects of the disclosure, the soybean plants, parts, seeds, and cells do not comprise a loss-of-function mutation in the soybean JAG1 and/or JAG2 gene.

“Breeding” refers to genetic manipulation of soybean varieties, including application of one or more agricultural and/or biotechnological tools, methods and/or processes to create useful new distinct varieties.

“Bulk” refers to a method of managing a segregating population during inbreeding that involves growing the population in a bulk plot, harvesting the self-pollinated seed of plants in bulk, and using a sample of the bulk to plant the next generation.

“Cell” refers to a soybean plant cell, whether isolated, in tissue culture, or incorporated in a plant or plant part. The soybean plant cell can be a cell, such as a somatic cell of the variety having the same set of chromosomes as the cells of the seed of the variety, or a cell that contains an edited genome or a locus conversion or transgene otherwise having the same or essentially the same set of chromosomes as the cells of the seed of the variety.

“Crossing” refers to mating, that is, fertilization by the union of two games from different soybean parent plants, such as by cross-pollinating to produce progeny. Crossing refers to a simple x by y cross or backcrossing, depending on context.

“Decreased seed weight” refers to decreased weight of a single seed or weight of a collection of seeds relative to a control seed or seed collection, or one or both parental varieties of the seed. A soybean breeder skilled in the art would understand that seed weight can be calculated in different ways, such as, for example, 100 seed weight, 1,000 seed weight, average single seed weight and seeds per pound. Decreased seed weight refers to a statistically significant decreased seed weight.

“Elite variety” refers to a soybean variety that is sufficiently homozygous and homogenous to be used for commercial grain production. An elite variety may also be used as source germplasm in further breeding.
“F1” refers to first generation progeny of the cross of two plants.
“F2” refers to second generation progeny of the cross of two plants.
“F3”refers to third generation progeny of the cross of two plants.
“F4”refers to fourth generation progeny of the cross of two plants.
“F5” refers to fifth generation progeny of the cross of two plants.
“F6” refers to sixth generation progeny of the cross of two plants.
“F7” refers to seventh generation progeny of the cross of two plants.
“F8”refers to eighth generation progeny of the cross of two plants.

“Locus” refers to a defined segment of DNA.

“Loss-of-function” refers to a mutation or allele of a gene which exhibits decreased gene function in comparison to an unmutated or wild-type allele of the gene. “Loss-of-function” mutations thus include mutations resulting in both null (i.e., amorphic) alleles and hypomorphic alleles (i.e., reduced by not eliminated function) of a gene.

“Nucleic acid” refers to an acidic, chain-like biological macromolecule consisting of multiple repeat units of phosphoric acid, sugar, and purine and pyrimidine bases. Nucleic acids include single stranded or double stranded nucleic acids, as specified, or contain portions of both double stranded or single stranded sequence. Nucleic acids include DNA (including genomic and cDNA), RNA (including mRNA and rRNA) or a hybrid, where the nucleic acid contains any combination of deoxyribo- and ribo-nucleotides, and any combination of bases.

“Phenotype” refers to one or more characteristics of a soybean plant. Phenotype can be observable to the naked eye, or by any other means of evaluation known in the art, e.g., decreased seed weight is evaluated by weighing a quantity of seed and seed size distribution is evaluated by quantifying the variation in seed size. In the case of seed weight and seed size distribution, such phenotypes are the result of several genes.

“Plant” means the soybean plant, in any of its stages of life, including reference to an immature or mature whole soybean plant, including a plant from which seed or grain or anthers have been removed. Any seed or embryo that will produce the plant is also considered to be the soybean plant.

“Plant part” includes a leaf, stem, root, root tip, anther, seed, grain, embryo, pollen, ovule, flower, cotyledon, hypocotyl, node, pod, flower, pistil, petiole, petal, shoot, stalk, tissue, protoplast, tissue culture, callus, clump, cell and the like. A plant part includes at least one cell, such as a somatic cell or a meristematic cell, of the soybean plant from which the plant part was obtained.

“Population” refers to a genetically heterogeneous collection of plants sharing a common genetic derivation.

“Progeny” refers to a soybean plant generated from a sexual reproduction from one or more soybean parent plants. A progeny plant may be obtained by selfing a single parent plant, or by crossing two parental plants and includes selfings as well as the F1 or F2 or still further generations. An F1 is a first-generation offspring produced from parents at least one of which is used for the first time as donor of a characteristic or trait, while offspring of second generation (F2) or subsequent generations (F3, F4, and the like) are specimens produced from selfings or crossings of F1s, F2s and the like. An F1 can thus be (and in some embodiments is) a hybrid resulting from a cross between two true breeding parents (the phrase “true-breeding” refers to an individual that is homozygous for one or more traits), while an F2 can be (and in some embodiments is) an offspring resulting from self-pollination of the F1 hybrids.

“Seed size distribution” refers to the seed sizes that are present in a collection of seeds and the frequency or proportion of certain sizes in the collection of seeds.

“Seeds per pound” is the number of soybean seeds per pound.

“Seed yield” is the dry yield in bushels/acre of the grain at harvest.

“Soybean” means Glycine max and includes all plant varieties that can be bred with soybean, including wild soybean species.

“Variety” refers to a substantially homozygous soybean line and minor modifications thereof that retains the overall genetics of the soybean line including but not limited to a subline, a locus conversion, a mutation, a transgenic, or a somaclonal variant. Variety includes seeds, plants, plant parts, and/or seed parts of the instant soybean variety.

An aspect of the disclosure is the development of an elite soybean variety having decreased soybean seed weight compared to one or more of the parents of the elite soybean variety. Pedigree breeding is an exemplary method of developing such an elite soybean variety and includes a step of crossing two parent varieties and generating segregating populations that undergo multiple generations of self-pollination and selection for decreased seed weight and seed size distribution, until a set of derived varieties having decreased seed weight and substantially the same seed size distribution as one or both parents and that combine the desirable characteristics of both parents is obtained. Because pedigree breeding is based on complementation of characteristics, this method of pedigree breeding is efficient for breeding soybean for qualitative characteristics such as seed weight and seed size. The pedigree breeding method is appealing to breeders because it allows building better soybean varieties by putting together, in the same plant, good characteristics that were present in different materials. Because all parental crossings are controlled, the genealogy of each parental variety and their progeny is known.

To obtain a soybean plant having decreased soybean seed weight and a desired size, a first step is to cross a first soybean plant having a first seed weight with a second, different soybean plant having a second, lower seed weight. Any commercial or non-commercial soybean varieties can be used in the breeding program. In aspects of the disclosure, the second, different soybean plant does not comprise a naturally occurring mutation in an endogenous soybean gene which confers the decreased seed weight. In aspects of the disclosure, the second, different soybean plant does not comprise a loss-of-function mutation in the soybean JAG1 gene and/or JAG2 gene. The soybean JAG1 gene is set forth in the https internet site “soybase.org” under the gene identifier Glyma.20G116200.1 and is described in Cai et al. Plant Biotechnology Journal (2021) 19, pp. 1898-1900 doi: 10.1111/pbi.13673. The soybean JAG2 gene is set forth in the https internet site “soybase.org” under the gene identifier Glyma.10g273800 and is described in Cai et al. Plant Biotechnology Journal (2021) 19, pp. 1898-1900 doi: 10.1111/pbi.13673. Seed weight determination methods are known to those soybean breeders skilled in the art.

Soybean plants can be crossed by either natural or mechanical techniques. Natural pollination occurs in soybeans predominately by self-pollination. In either natural or artificial crosses, flowering and flowering time are an important consideration. Soybean is a short-day plant, but considerable genetic variation exists for sensitivity to photoperiod. The critical day length for flowering ranges from about 13 h for genotypes adapted to tropical latitudes to 24 h for photoperiod-insensitive genotypes grown at higher latitudes. Soybeans seem to be insensitive to day length for 9 days after emergence. Photoperiods shorter than the critical day length are required for 7 to 26 days to complete flower induction.

Soybean flowers typically are self-pollinated on the day the corolla opens. The stigma is receptive to pollen about 1 day before anthesis and remains receptive for 2 days after anthesis, if the flower petals are not removed. Filaments of nine stamens are fused, and the one nearest the standard is free. The stamens form a ring below the stigma until about 1 day before anthesis, then their filaments begin to elongate rapidly and elevate the anthers around the stigma. The anthers dehisce on the day of anthesis, pollen grains fall on the stigma, and within 10 h the pollen tubes reach the ovary and fertilization is completed. Self-pollination occurs naturally in soybean with no manipulation of the flowers. For the crossing of two soybean plants, it is typically preferable, although not required, to utilize artificial hybridization. In artificial hybridization, the flower used as a female in a cross is manually cross pollinated prior to maturation of pollen from the flower, thereby preventing self-fertilization, or alternatively, the male parts of the flower are emasculated using a technique known in the art. Techniques for emasculating the male parts of a soybean flower include, for example, physical removal of the male parts, use of a genetic factor conferring male sterility, and application of a chemical gametocide to the male parts.

Either with or without emasculation of the female flower, hand pollination can be carried out by removing the stamens and pistil with a forceps from a flower of the male parent and gently brushing the anthers against the stigma of the female flower. Access to the stamens can be achieved by removing the front sepal and keel petals, or piercing the keel with closed forceps and allowing them to open to push the petals away. Brushing the anthers on the stigma causes them to rupture, and the highest percentage of successful crosses is obtained when pollen is clearly visible on the stigma. Pollen shed can be checked by tapping the anthers before brushing the stigma. Several male flowers may have to be used to obtain suitable pollen shed when conditions are unfavorable, or the same male may be used to pollinate several flowers with good pollen shed.

Crossing a first soybean plant having a first seed weight with a second, different soybean plant having a second, lower seed weight results in the production of F1 hybrid soybean seed on the female designated soybean plant, i.e., the plant that is cross-pollinated with anthers from the other different male-designated soybean plant in step (a). In some embodiments, the first soybean plant is cross-pollinated with anthers from the second, different soybean plant in step (a). In other embodiments, the second soybean plant is cross-pollinated with anthers from the first soybean plant in step (a). In other words, in some embodiments, the first soybean plant is the designated male and in other embodiments, the first soybean plant is the designated female.

The F1 hybrid seed may be harvested in bulk and planted. F1 hybrid plants may be visually confirmed by using certain single locus traits such as pod color, flower color, pubescence color or herbicide resistance which indicate that the seed is truly a hybrid. F1 plants that lack the expected trait may be rogued.

One or more progeny soybean plants of a subsequent generation result from planting the F1 hybrid soybean seed, permitting the F1 soybean plants to self-pollinate or, for subsequent generations, permitting the F2, F3, F4, F5, F6, F7, F8, etc. soybean plants to self-pollinate to produce a further progeny plant, and then repeating the cycle by using the progeny plant of the further subsequent generation from step instead of the plant grown from the F1 soybean seed with sufficient inbreeding and selection to produce an elite inbred soybean plant with a decreased seed weigh and a desired seed size/seed size distribution. In some embodiments, the seed size/seed size distribution of the inbred soybean plant with a decreased seed weight phenotype and the seed size distribution of the first soybean plant are not substantially different.

More specifically, an F2 population may be produced by selfing one or several F1's. Selection of the best individuals may begin in the F2 population (or later depending upon the breeder's objectives); then, beginning in the F3, the best individuals in the best families can be selected. Replicated testing of families can begin in the F3 or F4 generation to improve the effectiveness of selection for traits with low heritability. At an advanced stage of inbreeding (i.e., F6, F7 and F8), the best lines or mixtures of phenotypically similar lines are tested for potential release as new elite, commercial soybean varieties.

It would be readily understood by one skilled in the soybean breeding arts that different methodologies are known to determine seed size and seed size distribution. For example, in one method, seed size and seed size distribution may be determined by using exemplary software (GrainScan, Seed Extractor, SmartGrain) based on the image values of analyzed soybean seed. Alternatively, in another method, seed size and seed size distribution may be determined by passing soybean seed through round-hole screen mesh diameters ranging from 10/64 inches to 22/64 inches beginning with the largest mesh diameter and progressing to smaller mesh diameters. The step of determining seed size and/or seed size distribution may be preceded with a soybean seed cleaning step. In some embodiments, the cleaning step can produce soybean seeds that are substantially free of split seeds and non-seed plant material. A Clipper Office Tester is an example of a device that may be used to perform the cleaning step.

In practice, the soybean seed is fed to the hopper of the Clipper Office Tester and is the spread evenly across the full width of the top screen. Soybean seed then moves across the top screen, which has openings larger than the seed itself. Large foreign material is “scalped” off while the good seed falls through the screen. The bottom screen can either sift or scalp. To set up for sifting, the bottom screen openings should be smaller than the soybean seed being cleaned. Trash, weed seeds and splits drop through the bottom screen while the good product passes over it. When scalping, the screen openings must be larger than the product itself. The large foreign material is “scalped” off while the good product falls through the screen. Next, the product is routed through a column of air from the bottom blast fan. This blast of air effectively removes any lightweight trash and dust that may have remained after screening. Good, clean soybean seed is finally discharged at the bottom of the air chamber into a clean seed box.

It would be readily understood by one skilled in the soybean breeding arts that different methodologies are known to determine seed weight. For example, seeds per pound is determined by obtaining a one pound sample of cleaned soybean seed using a calibrated scale and then manually counting the number of seeds contained in the one pound sample. The step of determining seeds per pound can be preceded with a soybean seed cleaning step. In some embodiments, the cleaning step can produce soybean seeds that are substantially free of split seeds and non-seed plant material. A Clipper Office Tester is an example of a device that may be used to perform the cleaning step.

Selection of soybean plants is not necessarily dependent solely on the phenotype of a plant and may be done in conjunction with genetic investigations. For example, one may utilize a suitable genetic marker which is closely genetically linked to a trait of interest. One of these markers may therefore be used to identify the presence or absence of a trait in the offspring of a particular cross, and hence may be used in selection of progeny for continued breeding. This technique may commonly be referred to as marker assisted selection. Any other type of genetic marker or other assay which is able to identify the relative presence or absence of a trait of interest in a plant may also be useful for breeding purposes. Procedures for marker assisted selection applicable to the breeding of soybeans are well known in the art. Such methods will be of particular utility in the case of recessive traits and variable phenotypes, or where conventional assays may be more expensive, time consuming or otherwise disadvantageous. Types of genetic markers which could be used in accordance with the disclosed methods include, but are not necessarily limited to, Simple Sequence Length Polymorphisms (SSLPs), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs), and Single Nucleotide Polymorphisms (SNPs). Genetic markers associated with soybean seed weight are known to those skilled in the art.

In addition to seed weight, seed size and seed size distribution, some of the criteria used to select the soybean variety in various generations of breeding include, for example, yield, lodging resistance, emergence, seedling vigor, disease tolerance, maturity, plant height, seed oil and protein content.

Once harvested, pods are typically air-dried at not more than 38° C. until the seeds contain 13% moisture or less, then the seeds are removed by hand. Seed can be stored satisfactorily at about 25° C. for up to a year if relative humidity is 50% or less. In humid climates, germination percentage declines rapidly unless the seed is dried to 7% moisture and stored in an air-tight container at room temperature. Long-term storage in any climate is best accomplished by drying seed to 7% moisture and storing it at 10° C. or less in a room maintained at 50% relative humidity or in an air-tight container.

The inventive methods can decrease the elite variety soybean seed weight at least 1%, at least 2%, 3%, 4%, 5%, 6%, 7%, 8%, 9%, at least 10% when compared to the first seed weight of the first soybean parent plant and/or the second seed weight of the second soybean parent plant.

Elite soybean plants and/or germplasm identified, produced or selected by the methods of this disclosure are also provided, as are any progeny and/or seeds and/or plant parts derived from a soybean plant or germplasm identified, produced or selected by these methods.

The present disclosure also provides a seed, a plant part, or a plant cell of the soybean plants of the present disclosure. In some embodiments, the plant cell is an embryo, a pollen or an ovule of the plants. In addition, the present disclosure also provides tissue culture of regenerable cells of the soybean plants. In some embodiments, the tissue culture is made from regenerable cells that are derived from embryos, protoplasts, meristematic cells, callus, pollen, leaves, anthers, stems, petioles, roots, root tips, fruits, seeds, flowers, cotyledons, and/or hypocotyls.

### EXAMPLE 1

Soybean breeding and selection is a continuous process in which beneficial characteristics of soybean plants are improved. Soybean is a self-pollinated legume with natural outcrossing of less than 0.5 to about 1%. As a result of its self-pollinating reproductive behavior, plant breeding procedures such as pedigree breeding and single seed descent are some of the more common procedures used to develop improved soybean varieties. These procedures involve making crosses or hybrids by hand pollination followed by selection, testing, and ultimately release of an improved soybean variety.

The pedigree method of soybean breeding consists of growing progeny of crosses through generations of self-pollination by growing rows of progeny of plants selected in each generation. The selection is based, at a minimum, on phenotypic characteristics, with the pedigree of each line maintained in the subsequent generations. This method is useful for evaluating progeny of crosses between phenotypically different parents, since it enables identification and removal of a large number of undesirable progeny in the early generations, leaving a high frequency of superior lines for final selection in the later generations.

In this example, seeds per pound is determined as follows. Seeds are first cleaned using clipper office tester cleaner with a 22/64″ round scalping screen over a 10/67″ oblong scalping screen to remove split seeds and non-seed plant material. A 1 lb subsample of cleaned seed is counted on an Old Mill Model 900-2 seed counter to determine the number of seeds per pound.

In this example, seed size distribution is determined as follows. Seeds are first cleaned using clipper office tester cleaner with a 22/64″ round scalping screen over a 10/67″ oblong scalping screen to remove split seeds and non-seed plant material. A 1,000 g subsample of cleaned seeds is then placed on the top screen of a stack of round screens in this order: 26/64″, 22/64″, 20/64″, 18/64″, 16/64″, 15/64″, 14/64″, 13/64″, 12/64″, 11/64″, an 10/64″. A solid cover is placed over the screens, and the seeds are shaken on a Seedburo screen shaker for 5 mins. Afterward, the seeds on each screen are weighed. The distribution of seed size for the sample is represented by the distribution of weights for each screen. Average seed size distribution would be determined by averaging the seed weights per screen from multiple 1,000 g subsamples.

The objective of this example is to breed a new, elite soybean variety that produces seeds with decreased seed weight compared to one or both parental varieties (the decreased seed weight may be a targeted lower seed weight value), Additionally, a further objective is for the new, elite soybean variety to produce seed with decreased seed weight with comparable seed size and/or seed size distribution relative to one or both parental varieties.

Stigmas on a commercial, high-yielding parental soybean variety designated INARIHS (the female soybean parent) having an average of 3,200 seeds per pound value are crossed with pollen from anthers on a second proprietary parental soybean variety designated INARILS (the male soybean parent) having an average of 2,800 seeds per pound value. INARILS seeds per pound is 12.5% lower than the INARIHS seed per pound. Seed size distribution for the INARIHS variety and the INARILS variety are measured and do not substantially vary. Both INARIHS and INARILS parents also have desirable traits, including high yield and disease resistance that would combine to create an elite variety. F1 seeds produced on the female INARIHS variety from the initial cross are harvested in bulk.

Next, F1 seed is planted in the field and the resulting F1 plants are rogued to remove parental off-types based on visual appearance. Remaining F1 plants are allowed to self-pollinate and F2 seed is harvested in bulk. Next, the F2 seed is planted in the field, the resulting F2 plants are allowed to self-pollinate and F3 seed is separately harvested from selected individual F2 plants that show desirable visual characteristics. Undesirable plants are discarded.

Next, the F3 seed is planted in a plant-to-row field design, with each plant progeny being grown in a single row of four meter length with plant to plant spacing of 10 cm. The twenty best F3 plants are selected from the cross the resulting F3 plants are allowed to self-pollinate and F4 seed is separately harvested from selected individual F3 plants that show desirable visual characteristics including, but not limited to, high pod number, upright architecture, lack of green stem, etc.

F4 seed is planted in a plant-to-row field design, the resulting F4 plants are allowed to self-pollinate and F5 seed is bulked by row from selected F4 rows that show desirable visual characteristics. At low seed volumes, seed weight can be evaluated using 100 seed weight and seed sizing via screening of multiple subsamples. As compared to the heavier-seeded parent and a reduced target seed weight of 10%, progeny with 10% lower 100 seed weight than the heavier-seeded parent and comparable seed size distribution determined by screening are selected and advanced to F5 progeny. F5 seed and future progeny seed are planted and self-fertilized and are successively evaluated at multiple locations with sufficient seed produced to evaluate 1,000 seed weight and seed sizing via screening of multiple subsamples per location as well as yield and other desirable agronomic characteristics. As compared to the heavier-seeded parent and seed weight target, progeny with lower 1,000 seed weight and comparable seed size distribution determined by screening are selected, with confirmation across a minimum of 1 additional year of testing. High performing lines with the targeted decreased seed weights of 10% and comparable seed size distributions achieve the breeding targets and are advanced toward commercialization.

All publications, patents, and patent applications mentioned in the specification are indicative of the level of those skilled in the art to which this disclosure pertains. All such publications, patents, and patent applications are incorporated by reference herein for the purpose cited to the same extent as if each was specifically and individually indicated to be incorporated by reference herein.

What is claimed:

1. A method to obtain a soybean plant having decreased soybean seed weight, the method comprising the steps of:
(a) crossing a first soybean plant having a first seed weight with a second, different soybean plant having a second seed weight that is lower than the first seed weight to produce F1 hybrid soybean seed;
(b) crossing a plant grown from the F1 hybrid soybean seed with itself to produce seed of a progeny plant of a subsequent generation;
(c) growing a progeny plant of a subsequent generation from the seed of the progeny plant of the subsequent generation and crossing the progeny plant of the subsequent generation with itself to produce a progeny plant of a further subsequent generation; and,
(d) repeating steps (b) and (c) using the progeny plant of the further subsequent generation from step (c) in place of the plant grown from the F1 soybean seed in step (b), where steps (b) and (c) are repeated with sufficient inbreeding and selection to produce an elite inbred soybean plant having decreased seed weight compared to the first seed weight.

2. The method of claim 1, where seed weight is selected from the group consisting of 100 seed weight, 1,000 seed weight, average single seed weight, and seeds per pound.

3. The method of claim 1, where decreased seed weight is a statistically significant decreased seed weight.

4. The method of claim 1, where steps (b) and (c) are repeated with sufficient inbreeding and selection to produce an elite inbred soybean plant having a decreased seed weight compared to the second seed weight.

5. The method of claim 1, where a stigma on the first soybean plant is cross-pollinated with anthers from the second, different soybean plant in step (a).

6. The method of claim 1, where a seed size distribution of seed on the elite inbred soybean plant and a seed size distribution of seed on the first soybean plant are substantially the same.

7. The method of claim 1, where the average seed size of seed on the elite inbred soybean plant and the average seed size of seed on the first soybean plant are substantially the same.

8. The method of claim 1, where a seed size distribution of seed on the elite inbred soybean plant and a seed size distribution of seed on both the first soybean plant and the second soybean plant are substantially the same.

9. The method of claim 1, where the soybean seed weight of the elite inbred soybean plant is from 1% to 10% less than the first seed weight of the first soybean plant.

10. The method of claim 1, where the selection step (d) includes phenotypic evaluation and selection for decreased seed weight and/or seed size distribution.

11. The method of claim 1, where the selection step (d) includes marker assisted evaluation and selection for the decreased seed weight, such as by genotyping progeny for the presence and/or absence of a molecular marker associated with seed weight.

12. The method of claim 1, where the selection step (d) includes using genetic markers to compare a genetic complement of a progeny plant with a genetic complement of the first soybean variety and/or the second soybean variety.

13. The method of claim 1, where the selection in step (d) is performed in at least two progeny generations selected from the group consisting of F3 progeny, F4 progeny, F5 progeny, F6 progeny, F7 progeny and F8 progeny.

14. The method of claim 1, where the selection step in step (d) is performed in at least three, four or five progeny generations.

15. The method of claim 1, wherein the second, different soybean plant does not comprise a loss-of-function mutation in the soybean JAG1 gene and/or JAG2 gene which confers the decreased seed weight and optionally wherein the second, different soybean plant does not comprise a naturally occurring mutation in an endogenous soybean gene which confers the decreased seed weight.

16. A soybean plant produced by the method of claim 1, wherein the soybean plant does not comprise a loss-of-function mutation in the soybean JAG1 or JAG2 gene and optionally wherein the soybean plant does not comprise a naturally occurring mutation in an endogenous soybean gene which confers the decreased seed weight.

17. A soybean seed, plant part or plant cell produced by the soybean plant of claim 16, wherein the soybean seed, plant part, or plant cell does not comprise a loss-of-function mutation in the soybean JAG1 or JAG2 gene and optionally wherein the soybean seed, plant part, or plant cell does not comprise a naturally occurring mutation in an endogenous soybean gene which confers the decreased seed weight.

18. The soybean plant part of claim 17, where the plant part is selected from the group consisting of a leaf, pollen, embryo, root, root tip, anther, pistil, flower, seed, pod, and stem.
